(19) United States
(12) Patent Application Publication
(10) Pub. No.: US 2019/0125594 A1
(43) Pub. Date: May 2, 2019

(54) MULTILAYER MATERIAL COMPRISING AT LEAST A LAYER OF NON-WOVEN FABRIC

(71) Applicant: Pantex International SPA A Socio Unico, Trezzano Rosa (IT)

(72) Inventor: Pietro Angeli, Trezzano Rosa (IT)

(21) Appl. No.: 16/098,787

(22) PCT Filed: May 2, 2017
(86) PCT No.: PCT/IB2017/052538

§ 371 Date: Nov. 2, 2018

(30) Foreign Application Priority Data
May 2, 2016 (IT) 102016000044721

(51) Int. Cl. A61F 13/511; B32B 7/05; B32B 5/02; B32B 5/26; B32B 3/26; A61F 13/15; A61F 13/512

(52) U.S. Cl. CPC A61F 13/51104; B32B 7/05; B32B 5/022; B32B 5/26; B32B 3/266; B32B 2307/726; A61F 13/15707; A61F 13/51121; A61F 13/5123; B32B 2555/02; A61F 13/15699

(57) ABSTRACT

A multilayer material is described comprising at least one layer of pre-tensioned non-woven fabric and a second layer connected to the first layer by means of connection regions. The connection regions comprise a plurality of primary patterns. Each primary pattern consists of at least one central connection region and two non-rectilinear connection regions arranged alongside the central region, but without contact between them and with the central region. The central connection region and the non-rectilinear connection regions form overall an arc region comprising a concave region.

### TECHNICAL FIELD

The present invention concerns a multilayer material comprising at least one layer of non-woven fabric.

### STATE OF THE ART

Multilayer structures are known suitable for use in absorbent sanitary articles.

By multilayer material in the context of the present invention we mean that the material comprises at least two distinct layers bonded together, for example, by means of lamination.

By absorbent sanitary article in the context of the present invention we mean all absorbent products such as, for example, disposable absorbent articles, sanitary towels, panty liners, interlabial devices, catamenial/medicinal/surgical tampons, nappies, incontinence pads, towels, medications, breastfeeding pads, underarm sweat guards, products for underwear, trousers and shorts, make-up remover wipes, mattress/bed/chair protector pads, absorbent swabs for animals and similar.

Below, by absorbent sanitary article we mean any one of the above-mentioned products.

Originally absorbent products were made of composite materials formed of a plurality of layers containing fabrics or fibres generally made of cotton which allowed body fluids to be absorbed.

Subsequently the structures were further developed, incorporating cellulose pulp which is able to absorb 5-6 times its weight. Nowadays, for example, the structures of sanitary articles and, in particular, of nappies, sanitary towels and incontinence pads, incorporate absorbent materials in gel such as polyacrylates in combination with cellulose fibres, for example, thus making it possible to market relatively fine multilayer structures.

Absorbent sanitary articles like children's nappies, sanitary towels or incontinence pads are typical applications.

For these purposes multilayer materials are used comprising a first layer in contact with the user's skin and called generally and here below topsheet, generally made of non-woven fabric. Below the topsheet, a further layer is optionally arranged adapted to acquisition and distribution of the liquid, followed by a layer of absorbent material and lastly a backsheet which must be waterproof.

The topsheet must be rapidly crossed by the liquids, but at the same time it must act as a barrier and avoid return of the liquids from the absorbent layer towards the user's skin, in particular it must avoid the surface in contact with the skin becoming wet and must maintain the feeling of dryness on the skin.

The more permeable the material composing the topsheet, the more efficiently it will allow liquids to pass through.

Furthermore, the topsheet is often required to be soft to the touch, since it is in contact with the user's skin, and also give the user a feeling of cushioning. It is furthermore desirable for the topsheet to have a resilience to dry and wet in all directions.

The thicker the layer of material, the more difficult it is for the liquid to permeate.

Said problem has been solved by perforating the topsheet to allow the liquids to pass through the layer and rapidly reach the liquid acquisition and distribution layer and the absorbent material below.

However, perforation of the topsheet to accelerate the passage of the liquids is obtained by crushing the layer of material forming the topsheet, reducing the thickness thereof and therefore reducing the softness of the topsheet.

To attempt to solve this problem, topsheets have been used consisting of special composite materials or different perforation methods have been used.

Generally, the topsheets are made of non-woven fabric.

Very thick materials produced with the so-called “air through bonded” technology, abbreviated to ATB, are frequently used as topsheets. The fibres that compose the strip of non-woven material are thermally bonded with a jet of hot air without contact with pressure cylinders, unlike what occurs with the more common carded thermal bonded materials.

In addition to being very expensive, the ATBs do not ensure a sufficient dryness effect, which is ideal for topsheets.

The commonest non-woven fabrics used as topsheets, although inexpensive, are no longer considered soft enough to the touch and therefore users are in search of alternatives that are more pleasant to wear.

To form topsheets having a greater cushioning effect, the non-woven fabric is formed by two rotating rollers, one female, i.e. with hollows, and one male, i.e. with reliefs corresponding to the hollows of the first roller so as to form 3D patterns or designs on the layer of final non-woven fabric, which give an impression of three-dimensionality and a volume or cushioning effect.

However, said counter-rotating rollers with male and female designs are extremely difficult and costly to produce.

Furthermore, said rollers tend to become easily misaligned and therefore the resulting design on the topsheet is often inaccurate and does not allow the desired result to be obtained. Furthermore, the speed of the machine for winding the rollers is considerably slowed down to increase the precision of the design, but in this way the production costs of the material increase exponentially.

The need is therefore felt for a multilayer material comprising a topsheet for absorbent sanitary articles, in particular for children's nappies, incontinence pads or sanitary towels, able to solve the above-mentioned problems and which can also be produced at a lower cost.

### SUMMARY OF THE INVENTION

The object of the invention is therefore to produce a multilayer material which is both soft, pleasant in contact with the skin and allows rapid passage of body fluids, but at the same time remains dry and can therefore be advantageously used as a topsheet for an absorbent sanitary article and has a resilience to dry and wet in all directions and an optimal cushioning effect.

According to the present invention the object is achieved by a multilayer material according to claim 1.

### BRIEF DESCRIPTION OF THE FIGURES

FIG. 1 is a photograph of a multilayer material comprising a layer of non-woven fabric according to the present invention;

FIG. 2 is a schematic representation of a portion of the multilayer material of FIG. 1; and

FIG. 3 is a section view of a projection of a cylinder adapted to form a semi-arcuate region along the axis III-III of the multilayer material of FIG. 2.

### DISCLOSURE OF THE INVENTION

In the context of the present invention, by layer we mean a mass of homogeneous material laid more or less uniformly over a surface.

According to the present invention it is advantageous to use the multilayer material as topsheet for an absorbent sanitary article.

In one aspect of the present invention, the multilayer material 1 comprises at least two layers. Preferably at least one of the two layers is a non-woven fabric 2.

More preferably the multilayer material comprises at least 3 layers.

Preferably the multilayer material comprises a second layer of non-woven fabric or at least one perforated layer, for example a perforated film.

Preferably the layer of non-woven fabric consists of a fibrous material in strip form, also known as web.

The process for producing a multilayer material 1 according to the present invention preferably comprises the steps of unwinding a first layer of non-woven fabric from a first roller at a first rotation speed, unwinding a second layer from a second roller at a second rotation speed, passing the first layer and the second layer through a third and a fourth roller, keeping one against the other and rotating at a third rotation speed and at a fourth rotation speed respectively.

Preferably the first rotation speed and the second rotation speed are different, the third roller is mainly smooth and the fourth roller has projections adapted to form connection regions 3 between the first layer 2 and the second layer of multilayer material. The projections are adapted to form connection regions 3 comprising a primary pattern consisting of at least one central region 5 and two non-rectilinear regions 6,7 arranged side-by-side but without contact between them and with the central region and forming overall an arc region 8 comprising a concave region 9.

In particular the first rotation speed is lower than the second rotation speed. In particular the fourth rotation speed is different from the third rotation speed.

Preferably, therefore, for the formation of the multilayer material, the two layers of identical or different materials are unwound at preferably differentiated speeds starting from two rollers and then passed between two rotating counter rollers, one of which is smooth and the other provided with projections.

The projections have preferably in section, a truncated pyramid shape, as shown in FIG. 3.

Preferably the roller or cylinder from which a first strip of non-woven fabric is unwound rotates at a speed lower than the speed at which the second layer is unwound, which will form the multilayer material so that when the strip of non-woven fabric which will form the first layer passes on the contact line between the two cylinders, it is pre-tensioned.

By pre-tensioned we mean that the two unwinding speeds are different and therefore there is a differential tensioning between the two layers of material.

Said pre-tensioning ensures a better result during processing of the material and, in particular, during connection of the strip or layer of non-woven material to the nearby layer in the following step of the process.

In a following step, the two layers are passed between two rotating rollers to form connection regions between them and so as to form a design that follows a regular geometry, also called pattern, on at least one strip of non-woven fabric.

The two rollers are kept in contact against each other and preferably have different peripheral speeds and can connect the layers both by means of pressure and by increasing the temperature.

In particular, and more preferably, the smooth cylinder has a lower speed than the cylinder with projections to provide further improved results in terms of the volume or cushioning effect.

The layer 2 of non-woven fabric is therefore preferably pre-tensioned in the machine direction before being combined or bonded to the following layer.

The machine direction or MD coincides with the direction in which the various layers of material are unwound to be processed.

In one aspect of the present invention, the projections on one of the rotating cylinders are adapted to form connection regions between the layer 2 of non-woven fabric and the layer below, whether formed of a layer of non-woven fabric or a perforated film.

The projections therefore have a form suited to producing connection regions adapted to form a pattern on the final layer of non-woven fabric.

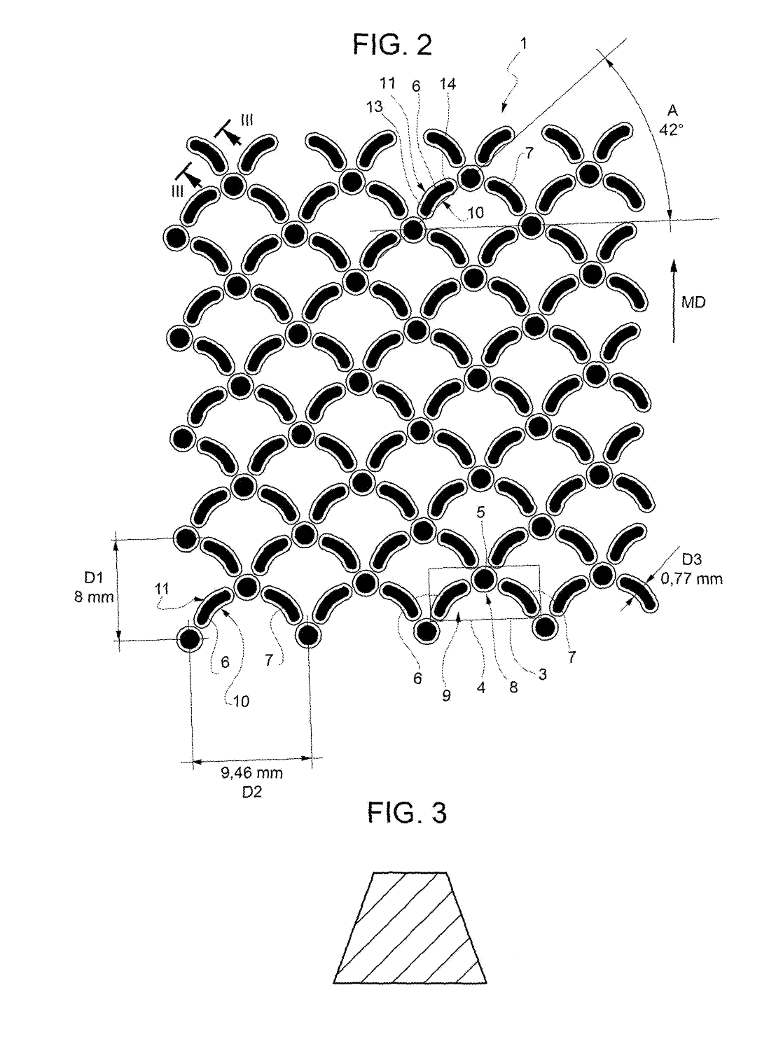

The connection or contact or bonding regions or areas 3, also commonly called “bonding areas”, on the non-woven fabric preferably comprise non-rectilinear regions 6, 7, more preferably semi-arcuate regions, for example similar to the shape of a comma or a kidney bean, like those schematically shown in FIG. 2.

More preferably the connection regions are formed of combinations of non-rectilinear regions, more preferably semi-arcuate regions side-by-side, or very close to one another, but without contact, and regions having various shapes, more preferably polygonal, circular, elliptical or also semi-arcuate.

The semi-arcuate regions preferably have a concave part and a convex part and more preferably the concave part faces towards the so-called machine direction.

More preferably the two semi-arcuate regions are arranged symmetrically around the central region.

Even more preferably, the line that joins the two terminal points of the concave part on the perimeter of the semi-arcuate region is arranged at an angle of between 30° and 60° with respect to the line perpendicular to the machine direction, even more preferably between 40° and 50°, for example 42° as shown in FIG. 2.

More preferably the connection areas are formed of a primary base pattern consisting of two semi-arcuate regions connected by a central region to form overall an arc which, during production of the material, has the concave part facing towards the machine direction or towards the two material unwinding rollers, i.e. the machine direction or MD.

By primary pattern we mean the simplest form which, when repeated, allows the overall pattern of the layer of non-woven fabric to be reproduced.

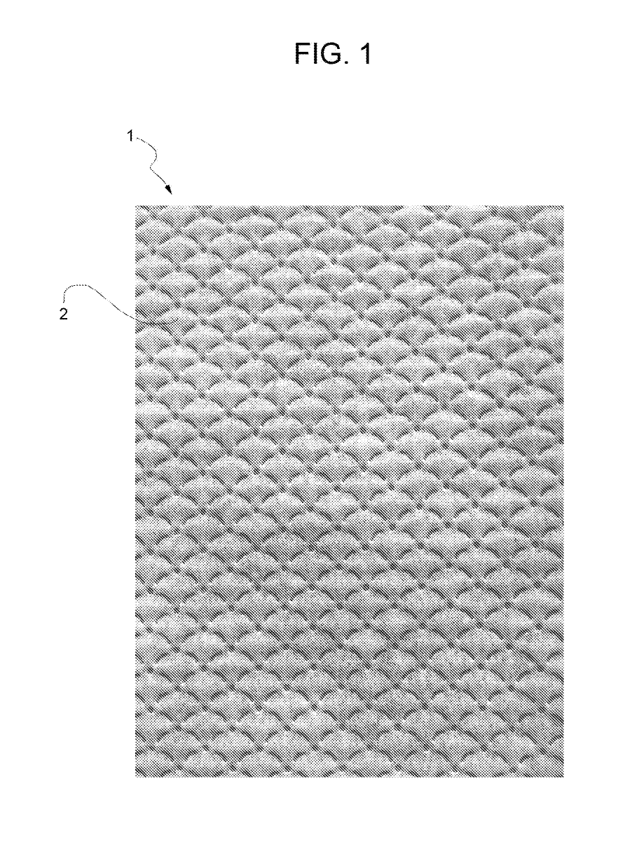

By overall pattern we mean the design which is formed overall on the layer of non-woven fabric by means of the connection areas as shown also graphically in FIG. 1 and schematically in FIG. 2.

Even more preferably the connection areas form overall a plurality of arc regions 8 each formed of a central region 5, with preferably circular or elliptical shape and arranged at the maximum height of the arc, also called “keystone”, and two semi-arcuate regions 6, 7 so as to form a semicircular arc or a C turned by 90° with a concave part 9, which is preferably facing downwards or towards the machine direction or MD during production of the material.

The central region 5 is central or comprised between the semi-arcuate regions 6, 7 and with reference to the axis perpendicular to the machine direction MD in FIG. 2.

More preferably, the central region 5 is formed of a region with regular shape, even more preferably a polygonal region and more preferably again a circular central region.

The central region 5 and semi-arcuate regions 6, 7 represent the connection or contact regions with the layer below, i.e. those regions that have been formed by pressing against the surface of the smooth roller, if necessary with the aid of a higher temperature. The connection or union between the two layers can be obtained not only via the method described of the two rotating rollers and therefore by means of pressure and if necessary high temperature to form the embossed regions, but also via other methods known in the sector.

An example of a real layer of non-woven fabric is shown in FIG. 1, while FIG. 2 shows a diagram of the same product. FIG. 3 shows a section of a projection on a roller adapted to form the semi-arcuate and central regions. The section is taken at a point adapted to form a semi-arcuate region at the axis III-III of FIG. 2.

In particular FIG. 1 shows the excellent cushioning effect on the finished product due to the shape of the connection areas which give the layer a three-dimensional effect and an effect of softness.

Preferably each arc region 8, which forms a primary pattern 4, is positioned alongside another arc region 8 to form an overall pattern in which each central region is surrounded by 4 semi-arcuate regions arranged in an X shape and the arc regions 8 follow one another regularly, more preferably symmetrically.

Considering the axis perpendicular to the machine direction, the entire non-rectilinear or semi-arcuate regions extend in an area preferably between 15° and 75°, more preferably between 30° and 60°, even more preferably between 40° and 50°. Preferably the connection areas 3 occupy a surface between 5 and 50% of the overall surface of the layer of non-woven fabric, more preferably between 10 and 30%.

Preferably the multilayer material 1 comprises two layers made of non-woven fabric. More preferably, both the non-woven fabrics have connection areas with the next layers having a pattern according to the present invention. Preferably the central points of the central regions are at a distance of between 2 and 20 mm, more preferably between 5 and 15 mm, even more preferably between 7 and 12 mm and they can be different in the direction perpendicular to that of the machine and in the machine direction. For example, they can be at a distance of 9.46 mm in the direction perpendicular to that of the machine and 8 in the machine direction.

Preferably the longest axis of the central regions is less than 5 mm, more preferably less than 2 mm.

Preferably the non-rectilinear or semi-arcuate regions have an overall length of less than 100 mm, more preferably less than 0.5 mm.

Preferably the non-rectilinear or semi-arcuate regions have a maximum width and a minimum width. Preferably the maximum width corresponds to the ends while the minimum width corresponds to the centre of the semi-arcuate region.

Preferably the minimum width or the only width is less than 2 mm, more preferably less than 1 mm, for example it can be 0.77 mm.

From an examination of the characteristics of the multilayer material produced according to the present invention, the advantages it offers are evident.

It has been ascertained that the multilayer material according to the present invention allows an excellent three-dimensional effect to be obtained and ensures an adequate feeling of softness in particular when used as a topsheet in an absorbent article.

Furthermore, in aesthetic terms the resulting pattern is similar to the capitonné effect in armchairs and therefore creates an even greater impression of softness.

1. A multilayer material (1) comprising at least one layer of non-woven fabric (2) and a second layer connected to said first layer by means of connection regions (3), characterised in that said connection regions comprise a plurality of primary patterns (4), each primary pattern consisting of at least one central connection region (5) and two non-rectilinear connection regions (6,7) arranged alongside said central region, but without contact between each other and with said central region, characterized in that said central connection region has a circular or elliptical shape and is central with respect to the two non-rectilinear connection regions (6, 7), said central connection region and said non-rectilinear connection regions form overall a semicircular arc region (8) comprising a concave region (9) and said central region is arranged at the maximum height of the arc.

2. The multilayer material (1) according to claim 1, characterized in that said second layer is a layer of non-woven fabric or a perforated film.

3. The multilayer material (1) according to claim 1, characterized in that it comprises a plurality of said arc regions (8) alongside one another in a regular succession of arc regions having the same dimensions.

4. The multilayer material (1) according to claim 1, characterized in that said non-rectilinear regions (6,7) are semi-arcuate regions having a concave portion (10) and a convex portion (11).

5. The multilayer material according to claim 1, characterized in that said concave portions (10) of said semi-arcuate regions (6, 7) are all facing towards the concave regions (9).

6. The multilayer material claim 1, characterized in that the line that joins the two terminal points (13, 14) of said concave regions (10) of said semi-arcuate regions (6, 7) is arranged at an angle ranging between 30° and 60° with respect to the line perpendicular to the unwinding direction of the material when wound on a roller or to the direction of the machine.

7. Absorbent sanitary article comprising a multilayer material according to claim 1.

8. A process for the manufacture of a multilayer material (1) comprising the steps of:
unwinding a first layer of non-woven fabric from a first roller at a first rotation speed
unwinding a second layer from a second roller at a second rotation speed
passing said first layer and said second layer through a third and a fourth roller held against each other and rotating at a third rotation speed and at a fourth rotation speed respectively, characterized in that:
the first rotation speed and the second rotation speed are different
said third roller is mainly smooth and said fourth roller has projections adapted to form connection regions (3) between said first layer (2) and said second layer of said multilayer material
said projections are adapted to form connection regions (3) comprising a primary pattern consisting of at least one central region (5) and two non-rectilinear regions (6,7) arranged side-by-side but without contact between them and with said central region and forming overall a semicircular arc region (8) comprising a concave region (9); said central connection region has a circular or elliptical shape and is central with respect to the two non-rectilinear connection regions (6, 7); said central connection region is arranged at the maximum height of the arc.

9. The process according to claim 8 characterized in that said first rotation speed is lower than said second rotation speed.

10. The process according to claim 8 characterized in that said fourth rotation speed is different from said third rotation speed.

11. The process according to claim 8 characterized in that a multilayer material is produced according to claim 2.
